(19) United States
(12) Patent Application Publication
(10) Pub. No.: US 2002/0156010 A1
(43) Pub. Date: Oct. 24, 2002

(54) Method of treating obesity in adult patients exhibiting primary insulin hypersecretion

(76) Inventor: Robert H. Lustig, San Francisco, CA (US)

Correspondence Address: Michael L. Goldman, NIXON PEABODY LLP, Clinton Square, P.O. Box 31051, Rochester, NY, 14603, US

(21) Appl. No.: 10/006,738

(22) Filed: Nov. 8, 2001

Related U.S. Application Data
(60) Provisional application No. 60/252,324, filed on Nov. 20, 2000.

(51) Int. Cl. A61K 38/31

(52) U.S. Cl. 514/12

(57) ABSTRACT

Methods of treating obesity in adult patients, reducing the caloric intake in an obese adult patient, and inhibiting insulin hypersecretion in an obese adult patient are disclosed. The methods are practiced by administering to an obese adult patient exhibiting primary insulin hypersecretion an effective amount of somatostatin, a somatostatin receptor agonist or its salt, or combinations thereof, under conditions effective to reduce the weight of the obese adult patient, reduce the caloric intake of the obese adult patient, or inhibit insulin hypersecretion by pancreatic β-cells of the obese adult patient.

This application claims benefit of U.S. Provisional Patent Application Serial No. 60/252,324, filed Nov. 20, 2000, which is hereby incorporated by reference in its entirety.

### FIELD OF THE INVENTION

The present invention generally relates to method of treating obesity, inhibiting insulin hypersecretion, and reducing the caloric intake in obese adult patients exhibiting primary insulin hypersecretion.

### BACKGROUND OF THE INVENTION

Obesity has reached epidemic proportions throughout the world. The prevalence of obesity (BMI>30 kg/m²) in the U.S. has risen from 12.8% to 22.5% during the last 20 years (Kuczmarski, et al., “Increasing prevalence of overweight in U.S. adults: The National Health and Nutrition Examination Surveys, 1960 to 1991,” JAMA, 272:205-211 (1994); Mokdad, et al., “The spread of the obesity epidemic in the United States, 1991-1998,” JAMA, 282:1519-1522 (1999); Bray, et al., “Current and potential drugs for treatment of obesity,” Endocrine Rev, 20:805-875 (1999)). Diet and exercise alone are frequently unsuccessful in ameliorating the obesity long-term (Luepker, et al., “Outcomes of a field trial to improve children's dietary patterns and physical activity,” JAMA, 275:768-776 (1996); Skender, et al., “Comparison of 2-year weight loss trends in behavioral treatments of obesity: diet, exercise, and combination interventions,” J Am Diet Assoc, 96:342-346 (1996); Bray, et al., “Treatment of obesity: an overview,” Diab Metab Rev, 4:653-679 (1988)), stressing the importance of metabolic and genetic components to this syndrome.

Obesity, defined as an excess of body fat relative to lean body mass, also contributes to other diseases. For example, this disorder is responsible for increased incidence of diseases such as coronary artery disease, hypertension, stroke, diabetes, hyperlipidemia, and some cancers (Nishina, et al., “Atherosclerosis in genetically obese mice: The Mutants Obese, Diabetes, Fat, Tubby, and Lethal Yellow,” Metab. 43: 554-558 (1994); Grundy, et al., “Metabolic and health complication of obesity,” Dis. Mon. 36:641-731 (1990)). Obesity is not merely a behavioral problem, i.e., the result of voluntary hyperphagia. Rather, the differential body composition observed between obese and normal subjects results from differences in both metabolism and neurologic/metabolic interactions. These differences seem to be, to some extent, due to differences in gene expression, and/or level of gene products or activity (Friedman, et al., “Molecular mapping of obesity genes,” Mammalian Gene 1:130-144 (1991); Barsch, et al., “Genetics of Body Weight Regulation,” Nature 404:644-651 (2000)).

Among risk factors and pathophysiological processes implicated in the etiology of obesity, the role of insulin remains controversial (Taylor, et al., “Insulin resistance or insulin deficiency: which is the primary cause of NIDDM,” Diabetes, 43:735-740 (1994); Ravussin, et al., “Insulin resistance is a result, not a cause of obesity: Socratic debate: the pro side,” In Progress in obesity research, Angel et al., Eds., London, Libbey, 173-178 (1996); Sims, et al., “EAH: Insulin resistance is a result, not a cause of obesity: Socratic debate: the con side,” In Progress in obesity research, Angel et al., Eds., London, Libbey, 587-592 (1996)). Insulin is the primary hormonal mediator of energy storage in humans (Marin, et al., “Glucose uptake in human adipose tissue,” Metabolism, 36:1154-1164 (1988)). Within the adipocyte, insulin regulates: Glut4 expression, acetyl-CoA carboxylase, fatty acid synthase, and lipoprotein lipase (Ramsay, “TG: Fat cells,” Endo Metab Clin NA, 25:847-870 (1996)). Most obese patients exhibit hyperinsulinemia (Lillioja, et al., “Exaggerated early insulin release and insulin resistance in a diabetes-prone population: a metabolic comparison of Pima Indians and Caucasians,” J Clin Endocrinol Metab, 73:866-876 (1991); Haffner, et al., “Increased insulin resistance and insulin secretion in non-diabetic African-Americans and Hispanics compared with non-Hispanic whites: the insulin resistance atherosclerosis study,” Diabetes, 45:742-748 (1996)); however, it is unclear whether this is a cause or effect of the obesity. It is also unclear whether insulin hypersecretion, decreased plasma insulin clearance, or insulin resistance is the crucial insulin defect. Acute glucose-stimulated insulin hypersecretion in insulin-sensitive adults predicts weight gain (Sigal, et al., “Acute post-challenge hyperinsulinemia predicts weight gain,” Diabetes, 46:1025-1029 (1997). In children, an augmented early postprandial insulin response precedes the development of obesity (LeStunff, et al., “Early changes in postprandial insulin secretion, not in insulin sensitivity, characterize juvenile obesity,” Diabetes, 43:696-702 (1994)). Conversely, fasting hyperinsulinemia has been shown to be a predictor of weight gain (Odeleye, et al., “Fasting hyperinsulinemia is a predictor of increased body weight gain and obesity in Pima Indian children,” Diabetes, 46:1341-1345 (1997); Zannolli, et al., “Hyperinsulinism as a marker in obese children,” Am J Dis Child, 147:837-841 (1993)).

In a rat model of obesity, lesioning of the ventromedial hypothalamus (VMH) causes excessive insulin secretion, hyperphagia, and intractable weight gain, which can be blocked by pancreatic vagotomy (Tokunaga, et al., “Effect of vagotomy on serum insulin in rats with paraventricular or ventromedial hypothalamic lesions,” Endocrinol, 119:1708-1711 (1986); Inoue, et al., “The effect of subdiaphragmatic vagotomy in rats with ventromedial hypothalamic lesions,” Endocrinol, 100:108-114 (1977); Bray, et al., “Manifestations of hypothalamic obesity in man: a comprehensive investigation of eight patients and a review of the literature,” Medicine, 54:301-333 (1975); Bray, et al., “Hypothalamic and genetic obesity in experimental animals: an autonomic and endocrine hypothesis,” Physiol Rev, 59:719-809 (1979); Bray, “Syndromes of hypothalamic obesity in man,” Pediatr Ann, 13:525-536 (1984)). Children who develop obesity secondary to cranial insult (Sorva, “Children with craniopharyngioma: early growth failure and rapid post-operative weight gain,” Acta Pediatr Scand, 77:587-592 (1988); Sklar, “Craniopharyngioma: endocrine sequalae of treatment,” Pediatr Neurosurg, 21:120-123 (1994)) exhibit insulin hypersecretion, and its suppression using octreotide (a somatostatin analog) promotes weight loss (Lustig, et al., “Hypothalamic obesity in children caused by cranial insult: altered glucose and insulin dynamics, and reversal by a somatostatin agonist,” J Pediatr, 135:162-168 (1999)). However, it is entirely uncertain whether obese adults, lacking such cranial insult, exhibit insulin hypersecretion and whether insulin suppression can induce weight loss.

The present invention is directed to overcoming these deficiencies in the art.

### SUMMARY OF THE INVENTION

A first aspect of the present invention relates to a method of treating obesity in adult patients which includes: administering to an obese adult patient exhibiting primary insulin hypersecretion an effective amount of somatostatin, a somatostatin receptor agonist or its salt, or combinations thereof, under conditions effective to reduce the weight of the obese adult patient.

A second aspect of the present invention relates to a method of reducing the caloric intake in an obese adult patient which includes: administering to an obese adult patient exhibiting primary insulin hypersecretion an effective amount of somatostatin, a somatostatin receptor agonist or its salt, or combinations thereof, under conditions effective to reduce the caloric intake of the obese adult patient.

A third aspect of the present invention relates to a method of inhibiting insulin hypersecretion in an obese adult patient which includes: administering to an obese adult patient exhibiting primary insulin hypersecretion an effective amount of somatostatin, a somatostatin receptor agonist or its salt, or combinations thereof, under conditions effective to inhibit insulin hypersecretion by pancreatic β-cells of the obese adult patient.

By the present invention, applicants have identified a subgroup of predominantly Caucasian subjects which exhibit primary insulin hypersecretion (PIH) as the pathogenesis of their obesity. Insulin suppression using the somatostatin analog octreotide resulted in loss of weight and fat mass. Insulin suppression may be a useful pharmacotherapeutic approach for this obesity subtype.

### BRIEF DESCRIPTION OF THE DRAWINGS

FIGS. 1 A-D are graphs which illustrate changes in weight (Δweight) and body mass index (ΔBMI) in 44 obese subjects treated with octreotide-LAR 40 mg IM q28 d for 24 weeks. Changes in (A) initial weight (Δweight) and (B) initial BMI (ΔBMI) were stratified post-hoc based on degree of BMI change. Subjects whose ΔBMI<−3 were high responders (HR, n=8, white squares); those whose ΔBMI was between −0.5 and −3 were low responders (LR, n=25, gray squares); and those whose ΔBMI>−0.5 were non-responders (NR, n=11, black squares). Error bars denote Standard Error of the Mean. (C) Δ weight and (D) ΔBMI were also stratified post-hoc based on race (Caucasians, n=27, white circles; Minorities, n=17, black circles). Although the difference between the races was not significant (ANOVA with repeated measures, P=0.058), there was a clear trend.

FIGS. 2A-H are graphs which illustrate excursions of c-peptide (A-D) and insulin (E-H) during oral glucose tolerance testing in 44 subjects with obesity. Curves for HR (a,e; white squares), LR (b,f; gray squares), and NR (c,g; black squares) are plotted both at Week 0 (solid lines) and at Week 24 (dashed lines). Error bars denote Standard Error of the Mean. ANOVA with repeated measures document significance of differences between the insulin curves for HR (E; P=0.001) and LR (F; P<0.001). Excursions of C-peptide (D) and insulin (H) are also grouped by Minorities (black circles) and Caucasians (white circles), both at Week 0 (solid lines) and at Week 24 (dashed lines). Significant differences were noted for both hormones (ANOVA with repeated measures) between the two timepoints for each race (P<0.001), although the difference of the curves between the races was not significant.

FIGS. 3A-H are graphs which illustrate correlations between ΔBMI (Week 24-Week 0) and changes in insulin indices during OGTT and fat mass by DEXA, during octreotide therapy in Minorities (A-D; black squares) and Caucasians (E-H; white squares). Significant differences are noted by equations and P values.

FIGS. 4A-H are graphs which illustrate a prediction of BMI response to octreotide based on pre-study insulin profile. This figure notes correlations between ΔBMI (Week 24-Week 0) during octreotide therapy, and pre-study insulin indices during OGTT and fat mass by DEXA in Minorities (A-D; black squares) and Caucasians (E-H; white squares). Significant differences are noted by equations and P values.

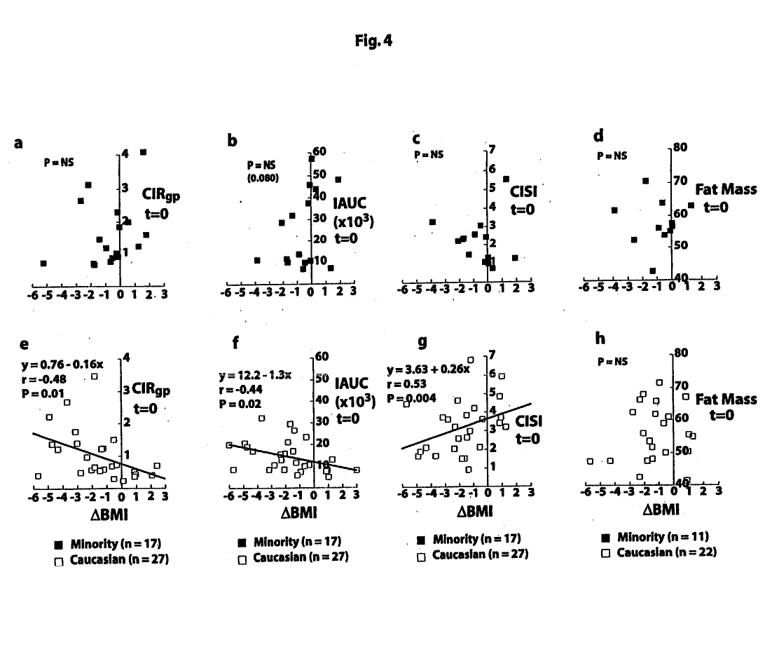

FIGS. 5A-B are graphs which illustrate correlations between changes in fat mass (ΔFat Mass) vs. changes in Insulin Area under the Curve (ΔIAUC; n=26), and changes in Composite Insulin Sensitivity Index (ΔCISI; n=30) during octreotide therapy. Significant differences are noted by equations and P values.

### DETAILED DESCRIPTION OF THE INVENTION

The present invention relates to methods of treating obesity in adult patients, reducing the caloric intake in an obese adult patient, and inhibiting insulin hypersecretion in an obese adult patient, each of which involves administering to an obese adult patient exhibiting primary insulin hypersecretion an effective amount of either somatostatin, a somatostatin receptor agonist or its salt, or combinations thereof, under conditions effective to reduce the weight of the obese adult patient, reduce the caloric intake of the obese adult patient, or inhibit insulin hypersecretion by pancreatic β-cells of the obese adult patient.

As used herein, the term “primary insulin hypersecretion” or “PIH” refers to individuals whose insulin dynamics are characterized by exaggerated insulin secretion during the upward glucose excursion of the oral glucose tolerance test (“OGTT”), while maintaining reasonably normal insulin sensitivity. Insulin secretion and insulin sensitivity can be measured objectively following administration of an OGTT (see Examples infra). Because PIH individuals exhibit high insulin secretion, they typically reach their glucose peak within about 60 minutes, more typically within about 30 minutes following administration of glucose. PIH individuals can be identified as those having a Corrected Insulin Response (“CIR”; a measure of beta-cell activity and insulin secretion) at the time of the glucose peak which is at least approximately 1.0 and, optionally, a composite insulin selectivity index (“CISI”; a measure of total body insulin sensitivity) of approximately 3.0 or less. Typically, though not exclusively, adult human patients exhibiting PIH are Causcasian.

Somatostatin is a cyclic, tetradecapeptide hormone having an amino acid sequence according to SEQ ID No: 1 as follows:

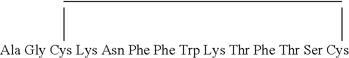

As used herein, a somatostatin receptor agonist refers to peptide and non-peptide compounds (i.e., peptidomimetics) which bind to a somatostatin receptor, preferably although not exclusively somatostatin receptor type 2 or somatostatin receptor type 5.

The somatostatin receptor agonist can be a somatostatin analog, which is intended to include straight-chain or cyclic peptides derived from naturally occurring somatostatin. Somatostatin analogs can include one or more amino acid residues which have been omitted and/or replaced by one or more other D- or L-amino acid residues and/or one or more other functional groups and/or one or more groups isosteric groups. In general, somatostatin analog is intended to describe all modified derivatives of the native somatostatin which have binding affinity in the nM range to at least one somatostatin receptor subtype. Exemplary somatostatin analogs include, without limitation, octreotide and lanreotide.

Octreotide is a cyclic octapeptide analog of somatostatin (D-phenylalanyl-L-cysteinyl-L-phenylalanyl-D-tryptophyl-L-lysyl-L-threonyl-N-[2-hydroxy-1(hydroxymethyl)propyl]-L-cysteinamide cyclic (2=>7)-disulfide). The preparation and use of octreotide is also well known (U.S. Pat. No. 4,395,403 to Bauer et al., which is hereby incorporated by reference in its entirety). Octreotide acetate is commercially available as Sandostatin-LAR® Depot (Novartis, East Hanover, N.J.). Other pharmaceutically acceptable salts can also be used. The amino acid sequence (SEQ ID No: 2) of octreotide is shown below.

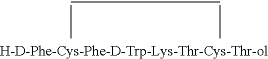

Lanreotide is a cyclic octapeptide analog of somatostatin. The preparation and use of lanreotide is also well known (U.S. Pat. No. 4,853,371 to Coy et al. and 5,411,943 to Bogden, each of which is hereby incorporated by reference in its entirety). Lanreotide acetate is commercially available as Somatuline® LA (Ipsen Biotech, Paris, France). Other pharmaceutically acceptable salts can also be used. The amino acid sequence (SEQ ID No: 3) of lanreotide is shown below.

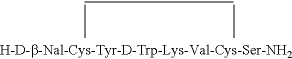

A number of other suitable somatostatin receptor agonists have been described in the art and disclosed, for example, in U.S. Pat. No. 6,270,700 to Ignatious, U.S. Pat. No. 6,127,343 to Ankersen et al., U.S. Pat. No. 6,117,880 to Guo et al., U.S. Pat. No. 6,083,960 to Ankersen et al., U.S. Pat. No. 6,063,796 to Yang et al., U.S. Pat. No. 6,057,338 to Yang et al., U.S. Pat. No. 6,025,372 to Yang et al., U.S. Pat. No. 6,020,349 to Ankerson et al., and U.S. Pat. No. 5,750,499 to Hoeger et al., each of which is hereby incorporated by reference in its entirety.

The obese adult patient exhibiting primary insulin hypersecretion is a mammal, preferably though not exclusively a primate, and in particular, a human.

The somatostatin or somatostatin receptor agonist (or combinations thereof) can be administered transdermally, parenterally, subcutaneously, intravenously, intramuscularly, intraarterially.

Suitable dosages of somatostatin or the somatostatin receptor agonist, or combinations thereof, can be readily determined by the care provider, based on efficacy of initial treatments, patient response, patient tolerance, etc. For subcutaneous injections, suitable dosages are on the order of about 1-100 μg/kg per day, preferably about 10-100 μg/kg per day. Total daily administration is typically about 200-1500 μg per day for subcutaneous delivery. For intramuscular injections, suitable dosages are on the order of about 20-60 mg/month or equivalent thereof, preferably about 25-55 mg/month. Dosages can, of course, vary from one somatostatin receptor agonist to another depending upon the in vivo half-life thereof. Determination of optimal ranges of effective amounts of each component is within the skill of the art.

### EXAMPLES

The following examples are provided to illustrate embodiments of the present invention but are by no means intended to limit its scope.

### PATIENTS AND METHODS

Inclusion and Exclusion Criteria: The study protocol was approved by the University of Tennessee Institutional Review Board, and all subjects gave written informed consent prior to eligibility confirmation. Inclusion criteria were ages 18-65 years and body mass index (BMI)≧35. The screening evaluation included a complete history and physical examination, and the following studies: Comprehensive Metabolic Panel (CMP), serum glucose 2 hours after an 75 gm oral glucose load, gallbladder ultrasound, and urine pregnancy test (if female). Exclusion criteria included gallstones, hypertension, diabetes mellitus (by ADA criteria), renal or liver disease, or use of chronic medications except for thyroid or estrogen supplementation. Subjects were evaluated every four weeks for 24 weeks.

Physical examination and laboratory evaluation: At each visit, subjects underwent physical examination, including vital signs, weight, height, and waist and hip circumference measurements (Bray, et al., “Treatment of obesity: an overview,” Diab Metab Rev, 4:653-679 (1988), which is hereby incorporated by reference in its entirety). A fasting venous blood sample was also obtained for CBC, CMP, lipids, HbA_{1c}, free T₄, TSH, IGF-1 (as a measure of growth hormone secretion) (Blum, et al., “Serum levels of insulin-like growth factor 1 (IGF-1) and IGF binding protein 3 reflect spontaneous growth hormone secretion,” J Clin Endocrinol Metab, 76:1610-1616 (1993), which is hereby incorporated by reference in its entirety), and leptin (as a surrogate marker of fat mass) (Guven, et al., “Plasma leptin and insulin levels in weight-reduced obese women with normal body mass index: relationships with body composition and insulin,” Diabetes, 48:347-352 (1999), which is hereby incorporated by reference in its entirety).

Dual-emission X-ray Absorptiometry (DEXA): Subjects were analyzed for total tissue, fat mass, and lean mass at Weeks 0 and 24 by DEXA, using a Lunar DPX-L machine (Madison, Wis.). Weight was limited to 137 kg, the upper limit for the table. Subjects received 0.06 mrem of radiation during the 40 minute scan. Auto width and length settings were utilized to reduce scan time and radiation exposure. The appropriate energy level was determined individually based on each subject's body habitus. The Week 24 scan was analyzed by comparison of regions of interest to the reference (Week 0) scan.

Oral Glucose Tolerance Test (OGTT): A three-hour OGTT was performed at Weeks 0 and 24 (Reaven, et al., “Insulin resistance and insulin secretion are determinants of oral glucose tolerance in normal individuals,” Diabetes, 42:1324-1332 (1993), which is hereby incorporated by reference in its entirety), after an overnight fast. Subjects drank 75 gm dextrose (Allegiance, MacGaw Park, Ill.), and blood samples were obtained at 0, 15, 30, 60, 90, 120, 150, and 180 minutes. The 1997 ADA diagnostic guidelines (“The expert committee on the diagnosis and classification of diabetes mellitus: Report of the expert committee on the diagnosis and classification of diabetes mellitus,” Diab Care, 20:1183-1197 (1997), which is hereby incorporated by reference in its entirety) were used to distinguish normal versus impaired glucose tolerance (IGT).

Chemical Analyses: Serum glucose during OGTT was measured by the glucose oxidase method (Kadish, et al., “A new and rapid method for the determination of glucose by measurement of rate of oxygen consumption,” Clin Chem, 14:116-119 (1968), which is hereby incorporated by reference in its entirety). Serum immunoreactive insulin (μU/ml) and C-peptide (ng/ml) levels from each OGTT sample were measured by standard double-antibody radioimmunoassay (RIA) (Linco Research; St Louis, Mo.). Leptin was measured by double antibody RIA, and IGF-1 by competitive binding RIA (Endocrine Sciences; Calabasas Hills, Calif.). All other laboratory studies were performed by Memphis Pathology Laboratory (Memphis, Tenn.).

Indices of insulin dynamics: The (a) Corrected Insulin Release at the glucose peak (CIRgp) (Sluiter, et al., “Glucose intolerance and insulin release, a mathematical approach. Assay of the beta cell response after glucose loading,” Diabetes, 25:241-244 (1976), which is hereby incorporated by reference in its entirety) is an index of β-cell activity. The (b) Fasting Insulin (FI), and (c) Composite Insulin Sensitivity Index (CISI) are measures of peripheral insulin sensitivity (Matsuda, et al., “Insulin sensitivity indices obtained from oral glucose tolerance testing: comparison with the euglycemic insulin clamp,” Diab Care, 22:1462-1470 (1999), which is hereby incorporated by reference in its entirety). The (d) Insulin Area under the Curve (I AUC) (Toft, et al., “Insulin kinetics, insulin action, and muscle morphology in lean or slightly overweight persons with impaired glucose tolerance,” Metabolism, 47:348-354 (1998), which is hereby incorporated by reference in its entirety) is a measure of the magnitude of the insulinemia. The (e) molar ratio of area under the c-peptide curve/I AUC (CP/I AUC) (Meistas, et al., “Hyperinsulinemia of obesity is due to decreased clearance of insulin,” Am J Physiol, 245:E155-E159 (1983), which is hereby incorporated by reference in its entirety) estimated insulin clearance during the OGTT.

$$CIRgp = \frac{Igp \times 100}{[ ( Ggp ) \times ( Ggp - 70 ) ]} \begin{matrix}where Igp = insulin at glucose peak , \\ Ggp = glucose at peak \end{matrix}$$

$$CISI = \frac{10000}{[ ( FI \times FBG ) \times {( mean insulin ( 0 –120 min ) \times mean glucose ( 0 –120 min ) ]}^{1 / 2}}$$

$$where FBG = fasting blood glucose$$

$$CP / I AUC = \frac{C \text{-} peptide AUC}{I AUC}$$

Statistics: All data analyses were performed using the SAS system (Cary, N.C.). Descriptive statistics are reported as mean and standard error of the mean (SEM) for continuous data and frequency and percent for categorical data. Area under the curve (AUC) was calculated by the trapezoidal method (Tallarida, et al., Manual of Pharmacologic Calculations with Computer Programs, Springer-Verlag, New York,” 77-81 (1986), which is hereby incorporated by reference in its entirety). Change scores for continuous data were computed by subtracting measures at Week 0 from Week 24. For one analysis, data were grouped into three categories of response based on BMI change: 8 high responders (ΔBMI<−3), 25 low responders (−3≦ΔBMI ≦−0.5), and 11 nonresponders (ΔBMI>−0.5). A second analysis grouped the data by race. The 15 African-Americans and 2 Hispanics were statistically indistinguishable; they are hereafter grouped as Minorities (39%). There were 27 Caucasians (61%). Statistical analyses applied to the data consisted of Pearson Chi-square, Pearson correlation, t-test, analysis of variance, ANOVA with repeated measures, and multi-variable linear regression. P-values less than or equal to 0.05 were considered significant, although trends (0.05<P<0.1) are also listed.

### Example 1

### Treatment of Adults Exhibiting Primary Insulin Hypersecretion with Octreotide

Subjects were treated with six injections of octreotide-LAR (Sandostatin-LAR® Depot; Novartis, East Hanover, N.J.) 40 mg IM q28 d from Weeks 0 to 20, given as two intragluteal 20 mg injections. Subjects were also treated with ursodeoxycholic acid (Actigall®; Novartis) 600 mg PO qd to prevent cholelithiasis (Williams, et al., “A double-blind placebo-controlled trial of ursodeoxycholic acid in the prevention of gallstones during weight loss after vertical banded gastroplasty,” Obes Surg, 3:257-259 (1993), which is hereby incorporated by reference in its entirety). Subjects were allowed to eat ad libitum, and neither dietary nor exercise interventions were recommended. Subjects checked their capillary blood glucose (CBG; Precision QID, Medisense, Needham, Mass.) three times a week, both before and 2 hr after a meal. Individual values were downloaded, and monthly averages of CBG were calculated at each visit to evaluate excursions of glucose in response to normal dietary intake.

Fifty-three subjects were recruited. Nine subjects (17%) dropped out during the study; 4 due to lack of weight loss during the first 4-20 weeks, and 5 for other reasons. Forty-four subjects completed the 24 weeks (Table 1 below).

TABLE 1
Clinical and Biochemical Characteristics of the Cohort

| | Mean | ±SEM | P |
|---|---|---|---|
| Clinical Variables | | | |
| Age (yr) | 38 | 1.3 | |
| Minorities/Caucasians | 17/27 | | |
| Females/Males | 39/5 | | |
| Weight Week 0 | 122.7 | 4.1 | |
| Weight Week 24 | 119.2 | 3.9 | |
| Δ Weight | −3.6 | 0.9 | <0.001 |
| BMI Week 0 | 44.3 | 1 | |
| BMI Week 24 | 43.1 | 1 | |
| Δ BMI | −1.2 | 0.3 | <0.001 |
| WHR Week 0 | 0.84 | 0.01 | |
| WHR Week 24 | 0.82 | 0.01 | |
| Δ WHR | −0.02 | 0.01 | 0.04 |
| Systolic BP Week 0 | 122.8 | 1.9 | |
| Systolic BP Week 24 | 124.5 | 1.9 | |
| Δ Systolic BP | 2.1 | 2.5 | NS |
| Diastolic BP Week 0 | 75.5 | 1.5 | |
| Diastolic BP Week 24 | 76.9 | 2 | |
| Δ Diastolic BP | 1.5 | 2.2 | NS |
| Insulin Indices | | | |
| CIRgp Week 0 | 1.43 | 0.16 | |
| CIRgp Week 24 | 0.62 | 0.09 | |
| Δ CIRgp | −0.84 | 0.1 | <0.001 |
| FI Week 0 | 20.02 | 1.78 | |
| FI Week 24 | 15.55 | 1.58 | |
| Δ FI | −4.54 | 1.59 | 0.007 |
| CISI Week 0 | 2.93 | 0.23 | |
| CISI Week 24 | 3.85 | 0.36 | |
| Δ CISI | 0.96 | 0.28 | 0.002 |
| I AUC Week 0 | 18282 | 2041 | |
| I AUC Week 24 | 12355 | 1662 | |
| Δ I AUC | −5423 | 1019 | <0.001 |
| CP/I AUC Week 0 | 0.1 | 0.01 | |
| CP/I AUC Week 24 | 0.1 | 0.01 | |
| Δ CP/I AUC | 0 | | NS |
| Biochemical Variables | | | |
| CBG Week 0 | 108 | 2.7 | |
| CBG Week 24 | 110 | 4.6 | |
| Δ CBG | 2.7 | 4.3 | NS |
| HbA1c Week 0 | 5.65 | 0.06 | |
| HbA1c Week 24 | 5.88 | 0.07 | |
| Δ HbA1c | 0.23 | 0.04 | <0.001 |
| FBG Week 0 | 93 | 1.5 | |
| FBG Week 24 | 111.3 | 1.9 | |
| Δ FBG mg/dl | 18.4 | 2.5 | <0.001 |
| IGF-1 Week 0 | 134.5 | 6.9 | |
| IGF-1 Week 24 | 109.6 | 3.4 | |
| Δ IGF-1 | −25.4 | 5.9 | <0.001 |
| Leptin Week 0 | 55.9 | 3.6 | |
| Leptin Week 24 | 41.9 | 2.7 | |
| Δ Leptin | −14 | 2.7 | <0.001 |
| TSH Week 0 | 1.52 | 0.12 | |
| TSH Week 24 | 1.18 | 0.12 | |
| Δ TSH | −0.34 | 0.11 | 0.005 |
| Free T4 Week 0 | 1.05 | 0.03 | |
| Free T4 Week 24 | 1.06 | 0.03 | |
| Δ Free T4 | 0.01 | 0.03 | NS |
| Cholesterol Week 0 | 183.5 | 5.2 | |
| Cholesterol Week 24 | 183.5 | 5.7 | |
| Δ Cholesterol | 0.1 | 3.3 | NS |
| Triglyceride Week 0 | 115.5 | 10.1 | |
| Triglyceride Week 24 | 112 | 8 | |
| Δ Triglyceride | −3.5 | 6.6 | NS |
| HDL Week 0 | 49.7 | 1.4 | |
| HDL Week 24 | 51.4 | 1.5 | |
| Δ HDL | 1.7 | 0.7 | NS |
| LDL Week 0 | 110.6 | 4.3 | |
| LDL Week 24 | 109.7 | 4.5 | |
| Δ LDL | −0.9 | 2.8 | NS |

Analysis by gender (5M, 39F) demonstrated no differences in response to octreotide. IGT was present in 14 subjects (32%). Seven subjects (16%) were receiving thyroxine supplementation.

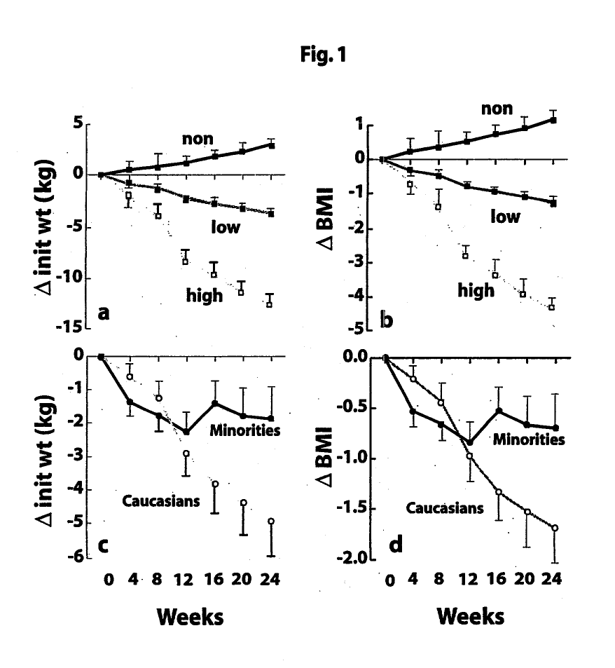

Weight, BMI, WHR: Weight, BMI and WHR were decreased by octreotide-LAR therapy in the entire cohort. Weight decreased by 3.6±0.9 kg (P<0.001), BMI decreased by 1.2±0.1 kg/m² (P<0.001), and WHR decreased by 0.02 ±0.01 (P=0.042). The magnitude of response was very broad (FIG. 1a,b). HR subjects lost 12.6±1.1 kg and BMI of −4.4±0.4, LR subjects lost 3.6±0.4 kg and BMI of −1.3±0.2, and NR gained 3.0 kg and BMI of +1.2±0.3 (P<0.001) (Table 2). The Caucasian population (FIG. 1c,d) lost 4.7±1.2 kg and BMI of −1.5±0.4 (P<0.001), and the Minority population lost 1.8±1.2 kg and BMI of−0.6±0.4, but the difference between the races was not significant (P=0.058) (Table 3).

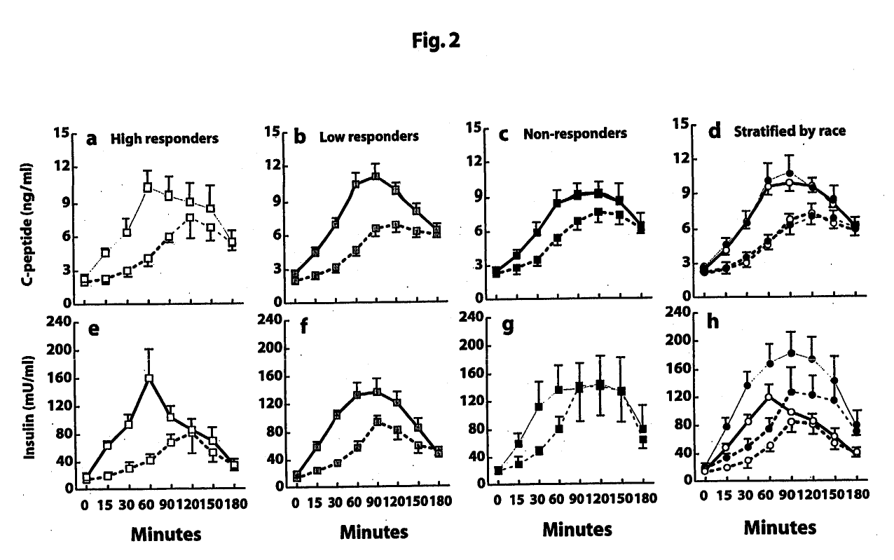

C-peptide and insulin curves: The C-peptide curves from the OGTT at Week 0 were indistinguishable among response strata (FIG. 2a-c), but the insulin curves were highly dissimilar (FIG. 2e-g). The HR insulin curve had a rapid ascending limb with a sharp peak, followed by a rapid decline. The NR insulin curve had a slow ascending limb with a plateau between 60 and 150 min. The LR insulin curve had components of both HR and NR curves, with a lack of an acute peak but with a shorter plateau. After 24 weeks of octreotide-LAR therapy, C-peptide suppression (FIG. 2a-c) was evident only in HR (P=0.001) and LR (P<0.001). Similarly, the insulin response was suppressed in HR (P=0.01) and LR (P<0.001). C-peptide curves were indistinguishable between races at both time points (FIG. 2d); however, Caucasians demonstrated decreased insulin responses versus Minorities, both at Week 0 (P=0.007) and Week 24 (P=0.043) (FIG. 2h).

Insulin indices: Octreotide-LAR suppressed CIRgp (P<0.001), decreased FI (P=0.007), and increased CISI (P=0.002) in the entire cohort (Table 1). CIRgp decreased amongst all response strata (HR P<0.001; LR P<0.0001; NR P=0.005) (Table 2 below). CISI increased in HR (P=0.006) and LR (P=0.001) only. IAUC declined in HR (P=0.001) and LR (P=0.001) only. CP/I AUC increased only in HR (P=0.01), and decreased in NR (P=0.01). Although CIRgp was suppressed by octreotide-LAR in both races (P<0.001) (Table 3 below), CIRgp was higher in Minorities versus Caucasians at both time points (Week 0 P=0.016; Week 24 P=0.009). FI was higher in Minorities at Week 0 (P=0.034), although at Week 24, the difference between the races was not significant (P=0.051). FI was suppressed by octreotide-LAR in both races, but only in Minorities was the decrease significant (P=0.045). CISI increased in both races during the study, but only in Caucasians was the increase significant (P=0.002). IAUC was higher in Minorities both at Week 0 (P=0.009) and at Week 24 (P=0.042). IAUC decreased in both races with therapy (Minorities P<0.001; Caucasians P=0.002). CP/I AUC was lower in Minorities both at Week 0 (P<0.001) and at Week 24 (P=0.002). CP/I AUC increased only slightly after insulin suppression in both races. However, for the change in each of these insulin indices during the treatment period, the difference between the races was not significant.

When Caucasians were analyzed separately (Table 2 below), CIRgp and IAUC were significantly greater in HR vs. NR at Week 0 (P=0.004 and 0.02, respectively). The decrease in both of these parameters was a function of the response strata (HR vs. LR: P=0.044 and 0.05; LR vs. NR: P=0.017 and 0.0003). Insulin sensitivity and clearance also improved in HR and LR over the study (P=0.03 and 0.004, respectively), but not in NR.

Leptin, DEXA, IGF-1: Serum leptin levels were indistinguishable between response strata or races at Week 0 (Tables 2-3 below). After 24 weeks of therapy, HR and LR demonstrated a significant decline in leptin (P<0.001), but NR showed no change (Table 2 below). Changes in leptin correlated with changes in BMI (P=0.003), but no racial differences were observed (Table 3 below).

The weight limit of the DEXA table (137 kg) precluded data acquisition in 11 subjects (25%). Our sample included 4 HR, 20 LR, and 9 NR subjects; and 11 Minority and 22 Caucasian subjects. Total tissue, fat mass, and lean mass were not different between response strata or races at Week 0 (Tables 2-3 below). After 24 weeks of therapy, total tissue decreased in HR and LR (P<0.001), and increased in NR (P=0.03). Fat mass also decreased in HR and LR (P=0.02 and P=0.01, respectively), and increased in NR (P=0.03). When analyzed by race (Table 3 below), total tissue and fat mass by DEXA were significantly decreased in Caucasians only (P=0.007 and P=0.03, respectively). Changes in BMI (Table 4 below) correlated with changes in total tissue (r=0.88, P<0.001), changes in fat mass (r=0.63, P<0.001), and changes in plasma leptin (r=0.58, P<0.001). Changes in lean mass decreased in HR (P=0.002) only; expected with the degree of weight loss seen in this response strata. Changes in lean mass did not correlate with changes in BMI.

At Week 0, IGF-1 levels were not different among response strata. Over the 24 weeks, IGF-1 decreased by 19% (P<0.001) in the entire cohort (Table 1); however, IGF-1 was suppressed the least in HR (P=0.025) (Table 2).

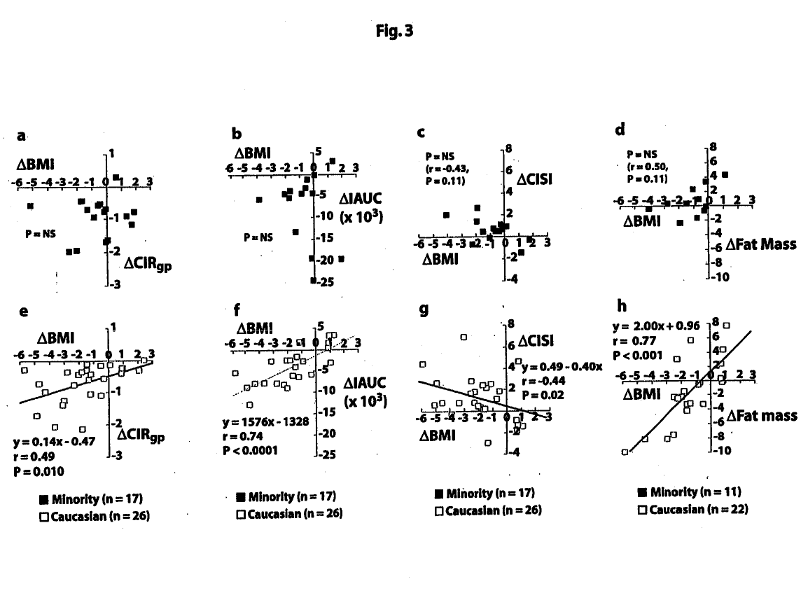

Correlation between BMI, insulin indices, fat mass, and CISI: For the entire cohort, change in BMI was negatively correlated with changes in CISI and CP/IAUC (P=0.003 and P=0.001, respectively), but were not correlated with changes in CIRgp and IAUC (P=NS and P=0.056, respectively). Changes in insulin indices in Minorities were not correlated with changes in BMI. When the Caucasian subpopulation was analyzed separately (Table 4a below, FIG. 3e-g), change in BMI correlated negatively with CISI and CP/I AUC (P=0.025 and P=0.002, respectively), and now exhibited a strong positive correlation with changes in CIRgp and IAUC (P=0.002 and P<0.001, respectively).

Prediction of weight loss: For the entire cohort, BMI change was not predicted by any insulin index or fat mass at Week 0. The correlation of FI and IAUC at Week 0 in Minorities with change in BMI showed a trend toward significance (P=0.056 and P=0.088; respectively), but in a positive direction. The small Minority sample size makes conclusions difficult. In Caucasians, there were significant correlations between pre-study CIRgp (P=0.011), CISI (P=0.005), IAUC (P=0.025), and CP/I AUC (P=0.002), versus changes in BMI while receiving octreotide. Pre-study fat mass was not predictive of BMI response.

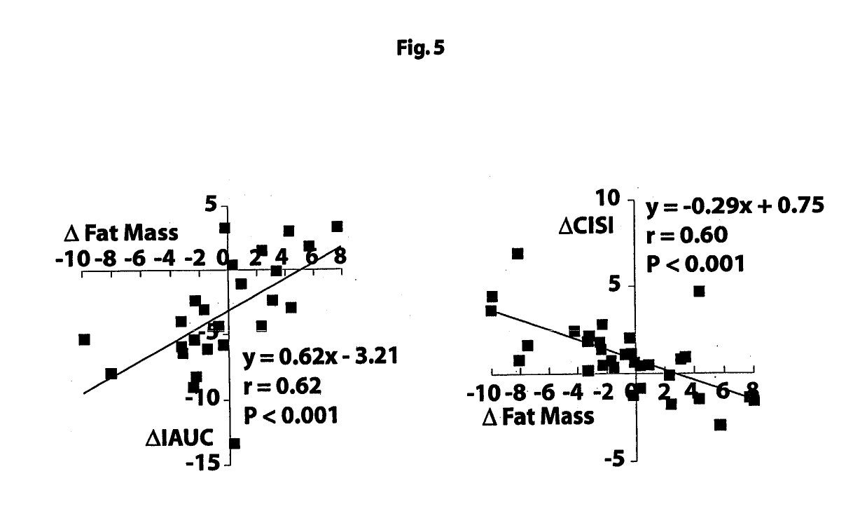

Correlation between insulin indices, fat mass, and CISI: Change in CISI was negatively correlated with change in BMI for the entire cohort (r=−0.45, P=0.003). The correlation coefficient for ΔCISI and ΔBMI for Minorities was of the same magnitude and direction as the entire cohort and Caucasians but was not significant due to the small sample size (n=17, r=−0.43, P=0.113). Change in CISI was also related to change in IAUC for Caucasians (r=−0.59, P=0.003) and marginally for the entire cohort (r=−0.32, P=0.058) but not for Minorities (r=0.01, P=NS). Changes in fat mass (FIG. 5) correlated with changes in IAUC (r=0.44, P=0.02) and CISI (r=−0.55, P=0.001). Multi-variable linear regression showed that for the entire cohort, the change in BMI was a significant independent predictor of change in CISI (regression coefficient=−0.35, P=0.026), but was again not significant for Minorities (reg coeff=−0.32, P=0.095), probably because of the small sample size. Interestingly, in Caucasians, ΔBMI was not a predictor of ΔCISI. Instead, ΔIAUC was the sole significant independent predictor of change in CISI (reg coeff=−0.00034, P=0.042), but neither fat mass nor BMI were significant predictors by themselves.

Safety: During the study, FBG increased by mean of 18 mg/dl (93±10 at Week 0, 111±12 mg/dl at Week 24 (P<0.001)). The percent of subjects with IGT increased from 32% before treatment to 73% at the end of the study. CBG at Week 0 was 108.2±3.0 mg/dl and 110.2±4.6 at Week 24 (P=NS). HbA_{1c} increased by 0.23±0.04% in the entire cohort (P<0.001), and there was no difference in the increase in HbA_{1c} based on IGT. No subject reported clinical symptoms suggesting diabetes mellitus or required diet or treatment for controlling blood glucose. Nineteen subjects complained of diarrhea at Week 4, which decreased to 3 by Week 24. Blood pressure, lipids and Free T₄ did not change appreciably (Table 1 above). TSH decreased from 1.52±0.8 IU/ml to 1.18±0.8 IU/ml with octreotide-LAR therapy (P=0.005). One subject developed cholelithiasis, but admitted to non-compliance with the preventative ursodeoxycholic acid therapy.

TABLE 2
Clinical and Biochemical Characteristics of the Cohort Grouped by BMI Response

| | High Responders | | Low Responders | | Non-responders | | | | |
|---|---|---|---|---|---|---|---|---|---|
| | (n = 8) | | (n = 25) | | (n = 11) | | Inter-response P | | |
| | Mean | ±SEM | Mean | ±SEM | Mean | ±SEM | HR v LR | HR v NR | LR v NR |
| Clinical Variable | | | | | | | | | |
| Age | 32.1 | 3.9 | 40.3 | 1.1 | 37.3 | 3.0 | 0.015 | NS | NS |
| Minorities/Caucasians | 1/7 | | 11/14 | | 5/6 | | | | |
| Sex Fh/M | 7/1 | | 22/3 | | 10/1 | | | | |
| Weight Week 0 | 139.7 | 14 | 117.5 | 4.7 | 122.4 | 6.3 | 0.046 | NS | NS |
| Weight Week 24 | 127 | 13.7 | 113.9 | 4.7 | 125.4 | 6.2 | NS | NS | NS |
| Δ Weight | −12.6 | 1.1 | −3.6 | 0.5 | +3.0 | 0.6 | <0.001 | <0.001 | <0.001 |
| P | <0.001 | | <0.001 | | <0.001 | | | | |
| BMI Week 0 | 47.4 | 3.3 | 42.4 | 1.1 | 46.4 | 2.2 | 0.07 | NS | NS |
| BMI Week 24 | 43 | 3.4 | 41.1 | 1.1 | 47.8 | 2.2 | NS | NS | 0.009 |
| Δ BMI | −4.4 | 0.4 | −1.3 | 0.2 | 1.4 | 0.2 | <0.001 | <0.001 | <0.001 |
| P | <0.001 | | <0.001 | | <0.001 | | | | |
| WHR Week 0 | 0.82 | 0.03 | 0.83 | 0.01 | 0.87 | 0.02 | NS | NS | NS |
| WHR Week 24 | 0.77 | 0.02 | 0.83 | 0.02 | 0.84 | 0.02 | NS | NS | NS |
| Δ WHR | −0.04 | 0.02 | 0 | 0.01 | −0.03 | 0.01 | 0.056 | NS | NS |
| P | 0.022 | | NS | | 0.06 | | | | |
| Laboratory Variable | | | | | | | | | |
| FBG Week 0 | 92.3 | 2.2 | 94.4 | 2.12 | 90.9 | 3.5 | NS | NS | NS |
| FBG Week 24 | 107 | 3.5 | 111 | 2.19 | 115.3 | 5 | NS | NS | NS |
| Δ FBG | +14.7 | 2.6 | +17 | 3.43 | +24.4 | 6.2 | NS | NS | NS |
| P | 0.014 | | <0.001 | | <0.001 | | | | |
| IGF-1 Week 0 | 152.1 | 21 | 133.3 | 9.1 | 124.8 | 10.6 | NS | NS | NS |
| IGF-1 Week 24 | 131.1 | 9.4 | 104.2 | 3.9 | 107.7 | 6.1 | 0.004 | 0.025 | NS |
| Δ IGF-1 | −24.4 | 16.5 | −29.4 | 8.3 | −17.1 | 9.2 | NS | NS | NS |
| P | NS | | 0.001 | | NS | | | | |
| Leptin Week 0 | 61.1 | 2.6 | 54.3 | 5.2 | 55.7 | 8.8 | NS | NS | NS |
| Leptin Week 24 | 34.1 | 3.3 | 38.6 | 3.8 | 55.2 | 4.1 | NS | 0.01 | 0.008 |
| Δ Leptin | −27 | 2.6 | −15.7 | 3.4 | −0.5 | 5.1 | 0.076 | 0.001 | 0.006 |
| P | <0.001 | | <0.001 | | NS | | | | |
| | High Responders | | Low Responders | | Non-responders | | | | |
| | (n = 4) | | (n = 20) | | (n = 9) | | Inter-response P | | |
| | Mean | ±SEM | Mean | ±SEM | Mean | ±SEM | HR v LR | HR V NR | LR V NR |
| DEXA Variables | | | | | | | | | |
| Total tissue Week 0 | 109 | 4.9 | 103.1 | 2.3 | 107.4 | 3.7 | NS | NS | NS |
| Total tissue Week 24 | 98.3 | 4.9 | 100.6 | 2.3 | 109.3 | 3.7 | NS | NS | NS |
| Δ Total tissue | −10.7 | 1.3 | −2.4 | 0.6 | 2 | 0.8 | <0.001 | <0.001 | <0.001 |
| P | <0.001 | | <0.001 | | 0.03 | | | | |
| Fat Mass Week 0 | 57.5 | 4.3 | 54.9 | 2 | 56.9 | 3.1 | NS | NS | NS |
| Fat Mass Week 24 | 52.6 | 4.1 | 52.3 | 2 | 60 | 3.1 | NS | NS | 0.047 |
| Δ Fat Mass | −5 | 2.1 | −2.5 | 0.9 | 3.1 | 1.4 | NS | 0.003 | 0.002 |
| P | 0.02 | | 0.01 | | 0.03 | | | | |
| Lean Mass Week 0 | 50.9 | 2.8 | 48.2 | 1.3 | 50.6 | 2.0 | NS | NS | NS |
| Lean Mass Week 24 | 45.7 | 2.7 | 48.3 | 1.3 | 49.3 | 2.0 | NS | NS | NS |
| Δ Lean Mass | −5.1 | 1.6 | 0.1 | 0.7 | −1.2 | 1.0 | NS | NS | NS |
| P | 0.002 | | NS | | NS | | | | |
| Insulin Indices | | | | | | | | | |
| CIRgp Week 0 | 1.54 | 0.24 | 1.32 | 0.17 | 1.62 | 0.51 | NS | NS | NS |
| CIRgp Week 24 | 0.45 | 0.11 | 0.49 | 0.07 | 1.01 | 0.3 | NS | 0.043 | 0.015 |
| Δ CIR gp | −1.09 | 0.17 | −0.86 | 0.12 | −0.6 | 0.29 | NS | NS | NS |
| P | <0.001 | | <0.001 | | 0.005 | | | | |
| FI week 0 | 18 | 2.5 | 20 | 2.3 | 21.6 | 4.7 | NS | NS | NS |
| FI Week 24 | 14.3 | 2.3 | 13.9 | 1.3 | 20.2 | 5.4 | NS | NS | NS |
| Δ FI | −3.7 | 1.7 | −6.2 | 1.7 | −1.5 | 5 | NS | NS | NS |
| P | NS | | 0.006 | | NS | | | | |
| CISI Week 0 | 2.76 | 0.38 | 2.7 | 0.26 | 3.41 | 0.65 | NS | NS | NS |
| CISI Week 24 | 4.49 | 0.85 | 3.97 | 0.46 | 3.15 | 0.79 | NS | NS | NS |
| Δ CISI | +1.73 | 0.55 | +1.31 | 0.33 | −0.26 | 0.61 | NS | 0.015 | 0.017 |
| P | 0.006 | | 0.001 | | NS | | | | |
| I AUC Week 0 | 16338 | 2819 | 18015 | 2222 | 20452 | 6508 | NS | NS | NS |
| I AUC Week 24 | 8918 | 2432 | 11149 | 1618 | 17759 | 5108 | NS | 0.035 | 0.032 |
| Δ I AUC | −7420 | 1054 | −6600 | 1331 | −1033 | 2399 | NS | 0.055 | 0.051 |
| P | 0.001 | | <0.001 | | NS | | | | |
| CP/I AUC Week 0 | 0.09 | 0.01 | 0.1 | 0.01 | 0.11 | 0.02 | NS | NS | NS |
| CP/I AUC Week 24 | 0.12 | 0.01 | 0.1 | 0.01 | 0.09 | 0.02 | NS | 0.07 | NS |
| Δ CP/I AUC | +0.03 | 0.01 | 0.01 | 0 | −0.02 | 0.01 | 0.028 | <0.001 | 0.008 |
| P | 0.002 | | NS | | 0.012 | | | | |
| | High Responders | | Low Responders | | Non-responders | | | | |
| Insulin Incides | (n = 7) | | (n = 14) | | (n = 6) | | Inter-response P | | |
| (Caucasian only) | Mean | ±SEM | Mean | ±SEM | Mean | ±SEM | HR v LR | HR V NR | LR V NR |
| CIRgp Week 0 | 1.6 | 0.25 | 1.1 | 0.17 | 0.48 | 0.27 | NS | 0.004 | 0.057 |
| CIRgp Week 24 | 0.5 | 0.12 | 0.42 | 0.09 | 0.33 | 0.13 | NS | NS | NS |
| Δ CIRgp | −1.2 | 0.17 | −0.71 | 0.12 | 0.2 | 0.18 | 0.04 | <0.001 | 0.01 |
| P | <0.001 | | <0.001 | | NS | | | | |
| FI week 0 | 18.6 | 3.8 | 18.7 | 2.7 | 11.6 | 4.1 | NS | NS | NS |
| FI Week 24 | 15 | 2.6 | 12.1 | 1.8 | 13.1 | 2.8 | NS | NS | NS |
| AFI | −3.6 | 2.3 | −6.6 | 1.6 | 1.5 | 2.5 | NS | NS | 0.01 |
| P | NS | | <0.001 | | NS | | | | |
| CISI Week 0 | 2.7 | 0.45 | 2.8 | 0.32 | 4.6 | 0.49 | NS | 0.007 | 0.005 |
| CISI Week 24 | 4.4 | 1.02 | 4.6 | 0.73 | 4 | 1.1 | NS | NS | NS |
| Δ CISI | 1.69 | 0.75 | 1.74 | 0.54 | −0.5 | 0.81 | NS | 0.05 | 0.02 |
| P | 0.03 | | 0.004 | | NS | | | | |
| I AUC Week 0 | 17091 | 2545 | 15736 | 1810 | 8048 | 2749 | NS | 0.02 | 0.03 |
| I AUC Week 24 | 9438 | 2657 | 10848 | 2749 | 9255 | 2870 | NS | NS | NS |
| Δ I AUC | −7653 | 1023 | −4888 | 851 | 1206 | 1105 | 0.05 | <0.0001 | 0.0003 |
| P | <0.001 | | <0.001 | | NS | | | | |
| CP/I AUC Week 0 | 0.09 | 0.01 | 0.1 | 0.007 | 0.14 | 0.01 | NS | 0.003 | 0.03 |
| CP/I AUC Week 24 | 0.12 | 0.01 | 0.11 | 0.01 | 0.11 | 0.01 | NS | NS | NS |
| Δ CP/I AUC | 0.03 | 0.02 | 0.001 | 0.007 | −0.02 | 0.01 | 0.05 | 0.001 | 0.05 |
| P | 0.009 | | NS | | 0.02 | | | | |

TABLE 3
Clinical and biochemical Characteristics of the cohort grouped by race

| | Minorities | | Caucasians | | |
|---|---|---|---|---|---|
| | (n = 17) | | (n = 27) | | Inter-race |
| | Mean | ±SEM | Mean | ±SEM | P |
| Clinical Variables | | | | | |
| Age | 37.7 | 1.7 | 39.9 | 1.7 | NS |
| sex F/M | 15/2 | | 24/3 | | |
| HR/LR/NR | 1/11/5 | | 7/14/6 | | |
| Weight Week 0 | 124.3 | 4.9 | 121.8 | 6 | NS |
| Weight Week 24 | 122.5 | 5 | 117 | 5.6 | NS |
| Δ Weight | −1.8 | 1.2 | −4.7 | 1.2 | NS |
| P | NS | | <0.001 | | |
| BMI Week 0 | 46 | 1.7 | 43.2 | 1.3 | NS |
| BMI Week 24 | 45.4 | 1.8 | 41.7 | 1.3 | 0.098 |
| Δ BMI | −0.6 | 0.4 | −1.5 | 0.4 | NS |
| P | NS | | <0.001 | | |
| WHR Week 0 | 0.86 | 0.02 | 0.82 | 0.01 | 0.077 |
| WHR Week 24 | 0.85 | 0.03 | 0.8 | 0.01 | 0.058 |
| Δ WHR | −0.01 | 0.02 | −0.02 | 0.01 | NS |
| P | NS | | 0.068 | | |
| Laboratory Variables | | | | | |
| FBG Week 0 | 92.4 | 2.8 | 93.5 | 1.8 | NS |
| FBG Week 24 | 111.5 | 2.8 | 111.3 | 2.6 | NS |
| Δ FBG | +19.3 | 4.5 | +17.9 | 3.1 | NS |
| P | <0.001 | | <0.001 | | |
| IGF-1 Week 0 | 138.1 | 8.7 | 132.4 | 10 | NS |
| IGF-1 Week 24 | 113.8 | 4.3 | 107 | 4.5 | NS |
| Δ IGF-1 | −25.1 | 8.9 | −25.5 | 8 | NS |
| P | 0.012 | | 0.001 | | |
| Leptin Week 0 | 60 | 7.5 | 53.3 | 3.7 | NS |
| Leptin Week 24 | 39.2 | 3.6 | 41.9 | 3.5 | NS |
| Δ Leptin | −16.3 | 5.5 | −11.4 | 2.8 | NS |
| P | <0.001 | | 0.003 | | |
| Insulin Indices | | | | | |
| CIRgp Week 0 | 1.92 | 0.3 | 1.13 | 0.15 | 0.016 |
| CIRgp Week 24 | 0.91 | 0.2 | 0.43 | 0.07 | 0.009 |
| Δ CIRgp | −1.02 | 0.2 | −0.72 | 0.11 | NS |
| P | <0.001 | | <0.001 | | |
| FI Week 0 | 24.9 | 3.1 | 17.2 | 2 | 0.034 |
| FI Week 24 | 19.4 | 3.4 | 13.1 | 1.3 | 0.051 |
| Δ FI | −5.38 | 3.7 | −4 | 1.3 | NS |
| P | 0.045 | | 0.053 | | |
| CISI Week 0 | 2.44 | 0.4 | 3.2 | 0.3 | NS |
| CISI Week 24 | 3.08 | 0.4 | 4.36 | 0.5 | 0.077 |
| Δ CISI | +0.61 | 0.3 | +1.17 | 0.4 | NS |
| P | NS | | 0.002 | | |
| I AUC Week 0 | 24875 | 4584 | 14478 | 1464 | 0.009 |
| I AUC Week 24 | 15943 | 3450 | 9963 | 1405 | 0.042 |
| Δ I AUC | −7573 | 2186 | −4115 | 900 | 0.07 |
| P | <0.001 | | 0.002 | | |
| CP/I AUC Week 0 | 0.08 | 0.01 | 0.11 | 0.01 | <0.001 |
| CP/I AUC Week 24 | 0.08 | 0.01 | 0.12 | 0.01 | 0.002 |
| Δ CP/I AUC | −0.01 | 0.01 | +0.01 | 0.01 | ND |
| P | NS | | NS | | |
| | Minorities | | Caucasians | | |
| DEXA | (n = 11) | | (n = 22) | | Inter-race |
| Variables | Mean | ±SEM | Mean | ±SEM | P |
| Total Tissue Week 0 | 106.5 | 3.0 | 103.8 | 2.2 | NS |
| Total tissue Week 24 | 105.4 | 3.0 | 101 | 2.2 | NS |
| Δ Total tissue | −1.1 | 1.3 | −2.7 | 0.9 | NS |
| P | NS | | 0.007 | | |
| Fat Mass Week 0 | 54.9 | 2.6 | 56.1 | 1.9 | NS |
| Fat Mass Week 24 | 55.8 | 2.6 | 53.7 | 1.9 | NS |
| Δ Fat Mass | 0.9 | 1.5 | −2.3 | 1 | 0.08 |
| P | NS | | 0.03 | | |
| Lean Mass Week 0 | 51.6 | 1.6 | 47.6 | 1.2 | NS |
| Lean Mass Week 24 | 49.7 | 1.6 | 47.2 | 1.2 | NS |
| Δ Lean Mass | −1.9 | 1.0 | −0.3 | 0.7 | NS |
| P | 0.08 | | NS | | |

TABLE 4
Correlations and predictors of BMI response

| Correlation | Δ BMI | | Predictor | Δ BMI | |
|---|---|---|---|---|---|
| Week 24-Week 0 | r | P | Week 0 | r | P |
| Δ CIRgp | | | CIRgp Week 0 | | |
| All | +0.24 | NS | All | −0.02 | NS |
| Minorities | −0.02 | NS | Minorities | +0.32 | NS |
| Caucasians | +0.57 | 0.002 | Caucasians | −0.48 | 0.01 |
| Δ CISI | | | FI Week 0 | | |
| All | −0.45 | 0.003 | All | +0.07 | NS |
| Minorities | −0.43 | NS | Minorities | +0.49 | 0.056 |
| Caucasians | −0.44 | 0.025 | Caucasians | −0.29 | 0.1 |
| Δ I AUC | | | CISI Week 0 | | |
| All | +0.32 | 0.056 | All | +0.23 | NS |
| Minorities | −0.01 | NS | Minorities | −0.14 | NS |
| Caucasians | +0.70 | <0.001 | Caucasians | +0.53 | 0.005 |
| Δ CP/I AUC | | | I AUC Week 0 | | |
| All | −0.51 | 0.001 | All | +0.10 | NS |
| Minorities | −0.32 | NS | Minorities | +0.46 | 0.088 |
| Caucasians | −0.60 | 0.002 | Caucasians | −0.44 | 0.02 |
| Δ Leptin | | | CP/I AUC Week 0 | | |
| All | +0.59 | 0.003 | All | +0.15 | NS |
| Minorities | +0.60 | 0.1 | Minorities | −0.27 | NS |
| Caucasians | +0.71 | 0.02 | Caucasians | +0.57 | 0.002 |
| Δ Fat Mass | | | | | |
| All | +0.63 | <0.001 | | | |
| Minorities | +0.50 | 0.1 | | | |
| Caucasians | +0.70 | <0.001 | | | |
| Δ Lean Mass | | | | | |
| All | +0.21 | NS | | | |
| Minorities | +0.53 | 0.09 | | | |
| Caucasians | +0.10 | NS | | | |

TABLE 5
Multiple variable linear regression: correlations and regressions vs. Δ CISI
Correlation vs. Δ CISI

| Variable | r | P |
|---|---|---|
| Δ BMI | | |
| All | −0.45 | 0.003 |
| Minorities | −0.43 | NS |
| Caucasians | −0.43 | 0.01 |
| Δ I AUC | | |
| All | −0.32 | 0.058 |
| Minorities | +0.01 | NS |
| Caucasians | −0.59 | 0.003 |
| Δ Fat Mass | | |
| All | −0.55 | 0.001 |
| Minorities | −0.69 | 0.02 |
| Caucasians | −0.52 | 0.01 |
| Δ Lean Mass | | |
| All | 0.14 | NS |
| Minorities | −0.33 | NS |
| Caucasians | 0.21 | NS |

Regression vs. Δ CISI

reg. coeff.

| | Δ BMI | Δ I AUC | Δ Fat Mass | r² |
|---|---|---|---|---|
| All | −0.35 | −0.00005 | −0.00014 | 0.37 |
| Minorities | −0.32 | −0.000007 | −0.00023 | 0.33 |
| Caucasians | +0.05 | −0.00034 | −0.00013 | 0.42 |

| P | All | Minorities | Caucasians |
|---|---|---|---|
| Δ BMI | 0.026 | 0.095 | NS |
| Δ Fat Mass | NS | NS | NS |
| Δ I AUC | NS | NS | 0.042 |

It is believed that this study represents the first evaluation of insulin hypersecretion in the pathogenesis of obesity and the first demonstration that octreotide is an effective therapeutic agent for suppression of insulin hypersecretion among obese individuals exhibiting PIH. The relation between the resultant insulin suppression and the weight and BMI response was also examined. A previous evaluation of the role of insulin suppression in obesity used diazoxide (Alemzadeh, et al., “Beneficial effect of diazoxide in obese hyperinsulinemic adults,” J Clin Endocrinol Metab, 83:1911-1915 (1998), which is hereby incorporated by reference in its entirety); however, this evaluation utilized a low-calorie formula diet in all patients, only lasted eight weeks, did not consider race as a co-variable, and did not examine differences between subject's responses and their baseline insulin profiles.

In the present study, it was discovered that insulin suppression using octreotide-LAR for 24 weeks promoted significant weight loss (mean 12.6 kg) and loss of fat mass (mean 5.0 kg) in 18% of an otherwise healthy subpopulation of adult obese subjects, and a small but significant loss of weight (mean 3.6 kg) and fat mass (mean 2.5 kg) in another 57%. This weight loss occurred slowly but without asymptote. Responders were primarily Caucasian, and showed a trend toward being younger, and with lower WHR; otherwise they were clinically indistinguishable from the rest of the cohort.

Octreotide promoted decreases in both leptin and fat mass by DEXA, suggestive of loss of adipose tissue. Although the weight limitation of the DEXA table could produce a sample bias, this bias is minimized, as the initial weight and BMI of the subject population was not predictive of weight loss or changes in fat mass. Nonetheless, changes in BMI with octreotide correlated with both changes in leptin and fat mass by DEXA (Table 4). Furthermore, the changes in fat mass with octreotide correlated with changes in insulin secretion and sensitivity (FIG. 5), suggesting that insulin was an important determinant in the pathogenesis of obesity in this cohort.

Octreotide binds to the somatostatin receptor-5 (SSTR₅) on the β-cell (Rohrer, et al., “Rapid identification of subtype-selective agonists of the somatostatin receptor through combinatorial chemistry,” Science, 282:737-740 (1998); Gordon, et al., “Cloning of the mouse somatostatin receptor subtype 5 gene: promoter structure and function,” Endocrinol, 140:5598-5608 (1999); Mitra, et al., “Colocalization of somatostatin receptor sst5 and insulin in rat pancreatic β-cells,” Endocrinol, 140:3790-3796 (1999), each of which is hereby incorporated by reference in its entirety) to inhibit the early phase of insulin secretion in a dose-dependent fashion (Bertoli, et al., “Dose-dependent effect of octreotide on insulin secretion after OGTT in obesity,” Hormone Research, 49:17-21 (1998); Giustina, et al., “Acute effects of octreotide, a long-acting somatostatin analog, on the insulinemic and glycemic responses to a mixed meal in patients with essential obesity: a dose-response study,” Diab Nutr Metab, 7:35-41 (1994); Lunetta, et al., “Long-term octreotide treatment reduced hyperinsulinemia, excess body weight and skin lesions in severe obesity with acanthosis nigricans,” J Endocrinol Invest, 19:699-703 (1996), each of which is hereby incorporated by reference in its entirety). Octreotide's effect is exemplified by the suppression of glucose-stimulated C-peptide and insulin excursions (FIG. 2). Despite these promising results, other potential mechanisms of octreotide action cannot be ruled out in the promotion of weight loss, such as: modulation of other GI hormones (Kiefer, et al., “The glucagon-like peptides,” Endocrine Rev, 20:876-913 (1999), which is hereby incorporated by reference in its entirety); slowing of gastric emptying and GI motility, with nutrient malabsorption (Simsolo, et al., “Effects of acromegaly treatment and growth hormone on adipose tissue lipoprotein lipase,” J Clin Endocrinol Metab, 80:3233-3238 (1995), which is hereby incorporated by reference in its entirety); direct effects on appetite (Lotter, et al., “Somatostatin decreases food intake of rats and baboons,” J Comp Physiol Psychol, 95:278-287 (1981); Levine, et al., “Peripherally administered somatostatin reduces feeding by a vagal mediated mechanism,” Pharmacol Biochem Behav, 16:897-902 (1982), each of which is hereby incorporated by reference in its entirety), or direct effects on the adipocyte (Simón, et al., “Characterization of somatostatin binding sites in isolated rat adipocytes,” Reg Peptides, 23:261-270 (1988); Campbell, et al., “Inhibition of growth hormone-stimulated lipolysis by somatostatin, insulin, and insulin-like growth factors (somatomedins) in vitro,” Proc Soc Exp Biol Med, 189:362-366 (1988), each of which is hereby incorporated by reference in its entirety). However, these other mechanisms seem less likely, as GI symptoms and appetite suppression were uniformly distributed throughout all response strata, and only those subjects who exhibited weight loss demonstrated insulin suppression, as exhibited by decreases in C-peptide and insulin excursions, and decreases in IAUC (Jiminez, et al., “Effects of weight loss in massive obesity on insulin and C-peptide dynamics: sequential changes in insulin production, clearance, and sensitivity,” J Clin Endocrinol Metab, 64:661-668 (1987), which is hereby incorporated by reference in its entirety). Furthermore, if alternate mechanisms other than insulin suppression were responsible for the weight loss, subjects receiving octreotide for acromegaly or other disorders would be expected to lose weight and fat mass; indeed long-term octreotide usage has a minimal positive effect on these parameters (Hansen, et al., “Body composition in active acromegaly during treatment with octreotide: a double-blind, placebo-controlled cross-over study,” Clin Endocrinol, 41:323-329 (1994), which is hereby incorporated by reference in its entirety).

During the study, the glucose excursion on OGTT worsened, and the frequency of IGT increased from 32% to 73%; however, the increase in %IGT was not reflected by increments in HbA_{1c} and CBG, which were small. Also, none of the HR subjects demonstrated IGT at Week 0 nor at Week 24. Perhaps delayed gastric emptying may account for the increase in glucose excursion in these subjects. In addition, HR subjects exhibited the least suppression of IGF-1 during treatment with octreotide.

There were clear racial discrepancies in weight response (FIG. 1c,d) and in insulin dynamics, both at Week 0 and at Week 24 (FIG. 2d,h; Table 3), to octreotide-LAR in this cohort. Minorities exhibited lower insulin sensitivity and decreased insulin clearance, along with increased β-cell activity and hyperinsulinemia, which were not explained by differences in weight, BMI, WHR, IGT, or diet (Svec, et al., “Black-white contrasts in insulin levels during pubertal development: the Bogalusa Heart Study,” Diabetes, 41:313-317 (1992); Jiang, et al., “Racial (black-white) differences in insulin secretion and clearance in adolescents: the Bogalusa Heart Study,” Pediatr, 97:357-360 (1996), each of which is hereby incorporated by reference in its entirety). Both their pre-study and post-treatment β-cell activities were elevated relative to Caucasians (Haffner, et al., “Increased insulin resistance and insulin secretion in non-diabetic African-Americans and Hispanics compared with non-Hispanic whites: the insulin resistance atherosclerosis study,” Diabetes, 45:742-748 (1996); Arslanian, et al., “Differences in the in vivo insulin secretion and sensitivity of healthy black versus white adolescents,” J Pediatr, 129:440-443 (1996), each of which is hereby incorporated by reference in its entirety). Despite equivalent suppression of insulin amplitude with therapy, they lost minimal weight and BMI. Conversely, Caucasians demonstrated higher insulin sensitivity and clearance at baseline. Their pre-study CIRgp and IAUC were lower than in Minorities, and their weight loss correlated with changes in these indices. Furthermore, in Caucasians only, pre-study and poststudy CIRgp and IAUC correlated with pre-study and poststudy leptin values (data not shown).

The pre-study CIRgp and IAUC in Caucasians predicted their weight loss in response to insulin suppression. Although both racial groups were hyperinsulinemic relative to the non-obese general population (Reaven, et al., “Insulin resistance and insulin secretion are determinants of oral glucose tolerance in normal individuals,” Diabetes, 42:1324-1332 (1993); Jiminez, et al., “Effects of weight loss in massive obesity on insulin and C-peptide dynamics: sequential changes in insulin production, clearance, and sensitivity,” J Clin Endocrinol Metab, 64:661-668 (1987); Sonnenberg, et al., “Splanchnic insulin dynamics and secretion pulsatilities in abdominal obesity,” Diabetes, 43:468-477 (1994), each of which is hereby incorporated by reference in its entirety), the difference in magnitude of these indices, and the differential weight responsiveness to what appears to have been equivalent suppression of C-peptide and insulin amplitude, connotes different etiologies and outcome between the races (Hafffer, et al., “Increased insulin resistance and insulin secretion in non-diabetic African-Americans and Hispanics compared with non-Hispanic whites: the insulin resistance atherosclerosis study,” Diabetes, 45:742-748 (1996); Dowling, et al., “Race-dependent health risks of upper body obesity,” Diabetes, 42:537-543 (1993); Albu, et al., “Systemic resistance to the antilipolytic effect of insulin in black and white women with visceral obesity,” Am J Phys, 277:E551-E560 (1999), each of which is hereby incorporated by reference in its entirety).

BMI response also correlated with changes in insulin sensitivity and clearance; a response noted by other investigators (Guven, et al., “Plasma leptin and insulin levels in weight-reduced obese women with normal body mass index: relationships with body composition and insulin,” Diabetes, 48:347-352 (1999); Rosenbaum, et al., “Obesity,” N Engl J Med, 337:396-407 (1997), each of which is hereby incorporated by reference in its entirety). Weight loss was associated with an improvement in CISI (Markovic, et al., “The determinants of glycemic responses to diet restriction and weight loss in obesity and NIDDM,” Diab Care, 21:687-694 (1998), which is hereby incorporated by reference in its entirety), but pre-study CISI did not predict weight loss (McLaughlin, et al., “Differences in insulin resistance do not predict weight loss in response to hypocaloric diets in healthy obese women,” J Clin Endocrinol Metab, 84:578-581 (1999), which is hereby incorporated by reference in its entirety). In fact, in Caucasians, CISI was instead a predictor of weight gain, as suggested by others (Sigal, et al., “Acute post-challenge hyperinsulinemia predicts weight gain,” Diabetes, 46:1025-1029 (1997), which is hereby incorporated by reference in its entirety). Of note is that within the Caucasian subpopulation only, improvement in insulin sensitivity was independently associated with insulin suppression, but not with weight loss or decrease in fat mass (Table 5). This suggests that the effect of weight loss on insulin sensitivity is mediated through the decrease in the insulinemia itself (Ratzmann, et al., “Effect of pharmacological suppression of insulin secretion on tissue sensitivity to insulin in subjects with moderate obesity,” Int J Obesity, 7:453-458 (1983), which is hereby incorporated by reference in its entirety). FI was only of value in the prediction of weight gain in Minorities (Table 4), and did not distinguish those subjects who responded with weight loss to insulin suppression.

Although this lacked a placebo control and lack of pharmacokinetic data which may explain differences in responsiveness, the correlation of BMI response with changes in β-cell activity and the prediction of BMI, leptin, and fat mass response based on baseline β-cell activity certainly indicates a primary role for insulin hypersecretion in the pathogenesis of obesity in our high-responders.

Changes in insulin secretion were found to correlate with weight loss and reductions in fat mass. Furthermore, pre-study β-cell activity predicted weight loss independent of any other intervention, but predominantly in Caucasians (88% of the HR group). These results provide evidence that a syndrome of Primary Insulin Hypersecretion (PIH), independent of insulin resistance, is a primary etiology of obesity, accounting for 26% of our Caucasian, and 18% of our entire cohort. PIH may be congenital or acquired (Lustig, et al., “Hypothalamic obesity in children caused by cranial insult: altered glucose and insulin dynamics, and reversal by a somatostatin agonist,” J Pediatr, 135:162-168 (1999); Van Assche, “Symmetric and asymmetric fetal macrosomia in relation to long-term consequences,” Am J Obstet Gynecol, 177:1563-1564 (1997), each of which is hereby incorporated by reference in its entirety), but in either case is characterized by a rapid and excessive early insulin response to OGTT, indicative of β-cell dysfunction. The insulin resistance seen in such patients appears to be secondary to the insulin hypersecretion (Sonnenberg, et al., “Splanchnic insulin dynamics and secretion pulsatilities in abdominal obesity,” Diabetes, 43:468-477 (1994); Ratzmann, et al., “Effect of pharmacological suppression of insulin secretion on tissue sensitivity to insulin in subjects with moderate obesity,” Int J Obesity, 7:453-458 (1983), each of which is hereby incorporated by reference in its entirety). Based on the above results, PIH can be considered a distinct entity with its own etiology, pathogenesis, and diagnosis.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A method of treating obesity in adult patients comprising:
administering to an obese adult patient exhibiting primary insulin hypersecretion an effective amount of somatostatin, a somatostatin receptor agonist or its salt, or combinations thereof, under conditions effective to reduce the weight of the obese adult patient.

2. The method according to claim 1, wherein said administering comprises intramuscular delivery.

3. The method according to claim 2, wherein the effective amount is about 20-60 mg/month.

4. The method according to claim 1, wherein said administering comprises subcutaneous delivery.

5. The method according to claim 4, wherein the effective amount is about 1-100 μg/kg per day.

6. The method according to claim 1, wherein a somatostatin receptor agonist is administered.

7. The method according to claim 6, wherein the somatostatin receptor agonist is a somatostatin analog.

8. The method according to claim 7, wherein the somatostatin analog is octreotide or lanreotide.

9. The method according to claim 6, wherein the somatostatin receptor agonist is an agonist of somatostatin receptor type 2 or somatostatin receptor type 5.

10. The method according to claim 1, wherein the patient is human.

11. A method of reducing the caloric intake in an obese adult patient comprising:
administering to an obese adult patient exhibiting primary insulin hypersecretion an effective amount of somatostatin, a somatostatin receptor agonist or its salt, or combinations thereof, under conditions effective to reduce the caloric intake of the obese adult patient.

12. The method according to claim 11, wherein said administering comprises intramuscular delivery.

13. The method according to claim 12, wherein the effective amount is about 20-60 mg/month.

14. The method according to claim 11, wherein said administering comprises subcutaneous delivery.

15. The method according to claim 14, wherein the effective amount is about 1-100 μg/kg per day.

16. The method according to claim 11, wherein a somatostatin receptor agonist is administered.

17. The method according to claim 16, wherein the somatostatin receptor agonist is a somatostatin analog.

18. The method according to claim 17, wherein the somatostatin analog is octreotide or lanreotide.

19. The method according to claim 16, wherein the somatostatin receptor agonist is an agonist of somatostatin receptor type 2 or somatostatin receptor type 5.

20. The method according to claim 11, wherein the patient is human.

21. A method of inhibiting insulin hypersecretion in an obese adult patient comprising:
administering to an obese adult patient exhibiting primary insulin hypersecretion an effective amount of somatostatin, a somatostatin receptor agonist or its salt, or combinations thereof, under conditions effective to inhibit insulin hypersecretion by pancreatic β-cells of the obese adult patient.

22. The method according to claim 21, wherein said administering comprises intramuscular delivery.

23. The method according to claim 22, wherein the effective amount is about 20-60 mg/month.

24. The method according to claim 21, wherein said administering comprises subcutaneous delivery.

25. The method according to claim 24, wherein the effective amount is about 1-100 μg/kg per day.

26. The method according to claim 21, wherein a somatostatin receptor agonist is administered.

27. The method according to claim 26, wherein the somatostatin receptor agonist is a somatostatin analog.

28. The method according to claim 27, wherein the somatostatin analog is octreotide or lanreotide.

29. The method according to claim 26, wherein the somatostatin receptor agonist is an agonist of somatostatin receptor type 2 or somatostatin receptor type 5.

30. The method according to claim 21, wherein the patient is human.
